(19) United States
(12) Patent Application Publication
(10) Pub. No.: US 2003/0113385 A1
(43) Pub. Date: Jun. 19, 2003

(54) Use of combinations of active agents consisting of antimicrobially active substances and plant extracts containing terpene in veterinary medicine

(75) Inventors: Werner Schleicher, Bingen am Rhein (DE); Ernst Salamon, Ingelheim am Rhein (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Vetmedica GmbH, Ingelheim (DE)

(21) Appl. No.: 10/219,180

(22) Filed: Aug. 15, 2002

Related U.S. Application Data
(63) Continuation of application No. 09/601,422, filed on Oct. 17, 2000, now abandoned.

(30) Foreign Application Priority Data
Feb. 2, 1998 (WO) PCT/EP98/00542

(51) Int. Cl. A61K 35/78; A61K 31/545; A61K 31/7048; A61K 31/43

(52) U.S. Cl. 424/725; 424/773; 514/29; 514/192; 514/200; 514/37

(57) ABSTRACT

Certain extracts of plants from the genera Leptospernum and Melaleuca can be used in the treatment of both mastitis and metritis to effectively reduce the amount of antibiotic employed, which extracts contain terpene.

The present invention relates to novel uses of combinations of active agents consisting of antimicrobially active substances and plant extracts containing terpene in veterinary medicine for the treatment of microbially caused diseases, especially mastitis and metritis in agricultural animals and small animals.

The aim of the present invention is to minimise the use of bactericidal or bacteriostatic agents required for treating bacterially caused diseases, since drugs of this kind involve or may be associated with undesirable side effects. For example, hypersensitivity reactions have been found when antibiotics are used in human medicine. In the veterinary medical field, in particular, the administration of large quantities of antibiotics to animals which are intended for consumption, or the products of which are intended for consumption, may lead to long waiting times, for example, to ensure that the drugs are not unintentionally taken by humans and thus promote the build-up of resistance to the pathogens, for example.

Surprisingly, it has now been found that the combination of antimicrobially active substances, preferably antibiotics, and most preferably ampicillin, cephalothin, penicillin G and spiramycin, which are typical examples of the amino penicillins (ampicillin), the cephalosporins (cephalothin), the benzyl penicillins (penicillin G) and the macrolide antibiotics (spiramycin) [M. Alexander, C. -J. Estler, F. Legler, Antibiotika und Chemotherapeutika, wissenschaftliche Verlagsgesellschaft mbH, Stuttgart 1995; Adam-Thoma, Antibiotika, Wissenschaftliche Verlagsgesellschaft mBH Stuttgart, 1994], with plant extracts containing terpene, preferably with extracts of plants of the genera Leptospermum and Melaleuca from the Myrtaceae family and most preferably with tea tree oil (extract of Melaleuca alternifolia) or with the oil of the cajuput tree (Melaleuca leucadendra) leads to a surprisingly high potentiation of the antimicrobial properties which significantly exceeds an additive effect and thus makes it possible to reduce the content of bactericidally or bacteriostatically active drug. In this way, on the other hand, the disadvantages mentioned above connected with the administration of antibiotics are avoided.

The preferred extracts of the leaves or plant parts of plants of the genera Leptospermum and Melaleuca, which occur naturally only in the subtropical coastal regions of New South Wales, are obtained by steam distillation or extraction. Most preferably, leaves of the Australia tea tree (Melaleuca alternifolia) are used as starting products.

Tea tree oil is virtually insoluble in water, but is readily miscible with most organic solvents, and consists of a mixture of many substances, including about 100 known ingredients. It is particularly rich in (+)-terpinen-1-ol and contains the following monoterpenes in smaller amounts [R. Saller and J. Reichling, Deutsche Apotheker Zeitung 135 (1995) 40 and lit. cit.]:

α-terpinene (about 10%), γ-terpinene (about 20%), terpinolene (about 4%), α-terpineol (3%), α-pinene, β-pinene, myrcene, α-phellandrene and 1.8-cineol as well as the sesquiterpenes aromadendrene, viridiflorene and δ-cadinene.

The bactericidal or bacteriostatic drug used may be any of the pharmaceutical compositions having a suitable activity spectrum as listed, in particular, in the 1996 Red List, Editio Cantor, Aulendorf/Württ. 1996, the contents of which are hereby referred to. The following antibiotics are mentioned as examples of particularly preferred active substances:

penicillins, particularly penicillin G, ampicillin and amoxycillin and bacampicillin, cephalosporins, β-lactam antibiotics, enzyme inhibitors such as β-lactamase inhibitors, e.g. oxacillin, cloxacillin, methicillin, or dihydropeptidase inhibitors, tetracyclines, such as oxytetracyclin, aminoglycosides—such as gentamycin, tobramycin, neomycin, canamycin, framycetin, streptomycin, etc., chloramphenicol, florphenicol and thiamphenicol, lincomycins and macrolide antibiotics, polypeptide antibiotics, quinolones and gyrase inhibitors, nitroimidazoles, as well as plant antibiotics such as percolate from Radix Umckaloabo.

For external use, in particular, the preferred compounds are tetracyclin, erythromycin, fusidic acid nebacetin, gentamycin, clindamycin, framycetin, neomycin, chloramphenicol, oxytetracyclin or sulphonamides.

Ampicillin, cephalothin, penicillin G and spiramycin are particularly preferred.

As is clear from the experimental findings, tea tree oil used on its own has no inhibitory effect on the growth of Staph. aureus in nutrient solution (cf. Experiment No. 1:

Effect of tea tree oil on the growth of Staphylococcus aureus in nutrient solution!).

The result is even clearer when the experiment is carried out in milk (cf. Experiment No. 2: Effect of tea tree oil on the growth of Staphylococcus aureus in normal milk!).

On the contrary, both experiments show that the growth of Staphylococcus aureus is rather promoted by the presence of tea tree oil. Correspondingly, the third experiment (sensitivity of Staphylococcus aureus to selected antibacterially active agents) demonstrates the inhibiting effect of typical examples of the categories of antibiotics mentioned hereinbefore. All the experiments demonstrate the expected inhibiting effect of these active substances both in nutrient medium and in milk.

The subsequent results of Experiments 5 to 7 demonstrate, for each of the antibacterially active substances investigated, that they have a significantly enhanced activity against both capsule-positive and capsule-negative species of Staphylococcus aureus.

The increase in the inhibiting effect particularly with a combination of tea tree oil and spiramycin is particularly striking.

The experimental findings described hereinafter provide clear evidence of promising treatment of microbially caused diseases by the use of combinations of active agents consisting of an antimicrobially active substance with a plant extract containing terpene, particularly for the treatment of mastitis or metritis in mammals, and particularly bacterially caused diseases in agricultural animals such as sheep, goats, horses, cattle or pigs, and in small animals such as dogs, cats and rabbits.

All the active agents mentioned may be used either on their own or in conjunction with other active substances and additionally with other excipients in the combination of active agents according to the invention. Similarly, the extracts containing terpene may be used as single extracts or as mixtures of extracts in the combination of active substances according to the invention.

The combinations of active substances according to the invention may be administered in the form of creams, ointments, lotions, water-in-oil or oil-in-water emulsions or aerosol foams. However, they may also be administered orally in the form of tablets, capsules, e.g. hard or soft gelatin capsules or coated tablets. The preparation of pharmaceutical forms of this kind is well-known per se from the prior art.

In veterinary medicine, the combination of active substances according to the invention may advantageously be used not only for treating metritis but particularly for treating mastitis in dairy cows and sows, the preferred preparations including, in addition to creams, ointments, lotions or emulsions, aerosol foams or a bolus.

Some pressurised foam compositions for the production of aerosol foams are mentioned hereinafter as selected examples of typical preparations. These compositions consist essentially of a so-called carrier, antioxidants for stabilising the components against the effects of oxygen, foam forming agents, emulsifiers, preservatives and a propellant gas, in addition to the plant extract containing terpene and the antimicrobially active substance.

Aerosol foams of this kind can be administered either directly as a fixed combination of tea tree oil with antibiotics or by the successive application of a desired antibiotic preparation (in the form of an ointment, foam, etc.) and a pressurised foam preparation containing tea tree oil on its own. This latter form of application may achieve better distribution in the target organ together with an increase in the activity (booster effect).

As examples of this, two of these “booster” compositions (3 and 4) are given hereinafter.

The carrier may be formed from water and/or oily components.

Suitable oily components are any of the active substances known from the prior art for the preparation of pharmaceuticals, such as, for example, vegetable oils, in particular, e.g. cotton seed oil, groundnut oil, maize oil, rapeseed oil, sesame oil and soya oil, or triglycerides of moderate chain length, e.g. fractionated coconut oil, or isopropylmyristate, -palmitate or mineral oils or ethyloleate.

The antioxidants used may be any of the antioxidants known from the prior art, preferably a-tocopherol, butylhydroxytoluene (BHT) or butylhydroxyanisole (BHA).

The foam-forming agents used may be any of those which are permitted under the drug licencing laws and known from the prior art, preferably polyoxyethylene sorbitanesters of various fatty acids (polysorbates).

The preferred emulsifiers used, apart from the emulsifiers known from the prior art, include polyoxyethylene derivatives of castor oil or polyoxyethylene alkylethers.

The above requirements also apply to the preservatives, the preferred ones being those selected from the group of the PHB esters, e.g. mixtures of PHB-methyl with PHB-propylesters, quaternary ammonium compounds such as benzalkonium chloride, phenol, chlorbutanol, chlorocresol, ethyl alcohol, thiomersal, phenyl-mercury salts such as nitrates, borates, etc., or benzoic and sorbic acid and the salts thereof.

Suitable propellant gases are all those which are licensed for use in the medical field and those which are known from the prior art, e.g. CO₂, N₂O, N₂, propane/butane mixtures, isobutane, chloropentafluoroethane (CClF₂-CF₃), octafluorocyclobutane (C₄F₆).

Some preparations by way of example follow the experimental section.

The invention described will now be illustrated by the Examples which follow. Various other embodiments will become apparent to the skilled person from the present specification. However, it is expressly pointed out that the Examples and description are intended solely as an illustration and should not be regarded as restricting the invention.

I. Experimental Results:

Preliminary Remarks:

The sensitivity tests and the experiments carried out with combinations of active agents—using the so-called Checkerboard design—in broth (nutrient medium) and milk, were carried out using microtitre plates. Three capsule-positive and three capsule-negative strains of Staphylococcus aureus were used. The so-called MIC value was determined by fluorometric methods (in milk).

The MIC value denotes the minimum concentration at which an inhibiting effect can be demonstrated. The concentration of the standard solution of the tea tree oil used was 4% vol/vol.

The highest concentration used in the tests was 1/10^{th} of the original standard solution, i.e. 0.4 vol-%.

The MIC in the following Examples is defined as the lowest concentration of active substance which inhibits a bacterially produced increase in turbidity—in the nutrient solution—or an increase in fluorescence—in milk.

FIC (fractionally inhibiting concentration)=

A/MICa, wherein A denotes the MIC value of the antibacterially active substance in the presence of the highest concentration of tea tree oil and MICa denotes the MIC value of the antibacterially active substance on its own.

Thus, an FIC value of <0.5 indicates an increase in activity by the tea tree oil (Ttoil), whereas FIC values of >1 imply an antagonism.

Miglyol [Fiedler, H. P., Lexikon der Hilfsstoffe für Pharmazie, Kosmetik und angrenzende Gebiete, 4^{th} Edition, Editio Cantor Verlag, Aulendorf 1996, Vol. II], as an emulsified standard solution containing 10% v/v, shows no inhibiting effect on the growth of S. aureus in milk (at a maximum concentration of 1%).

1 Effect of tea tree oil (Ttoil) on the growth of Staphylococcus aureus in nutrient solution (ISB).

The results are given as the average minimum inhibitory concentration from four repeats

MIC in ISB (mg/ml)

| | Capsule negative Staph. | | | Capsule positive Staph. | | |
|---|---|---|---|---|---|---|
| | aureus | | | aureus | | |
| | SaA | SaB | SaC | SaD | SaE | SaF |
| Ttoil | 4 | 4 | >4 | >4 | >4 | 2 |

2 Effect of tea tree oil on the growth of Staphylococcus aureus in normal milk. Each strain was tested four times.

MIC in milk (mg/ml)

| | Capsule negative Staph. | | | Capsule positive Staph. | | |
|---|---|---|---|---|---|---|
| | aureus | | | aureus | | |
| | SaA | SaB | SaC | SaD | SaE | SaF |
| Ttoil | >4 | >4 | >4 | >4 | >4 | >4 |

3 Susceptibility of Staphylococcus aureus to selected antibacterial agents in nutrient medium (ISB) and milk. The results are given as the average minimum inhibitory concentration (MIC) (+standard deviation) of three capsule positive or three capsule negative strains. Each strain was tested 4 5 times.

MIC in ISB (μg/ml)

| | Capsule negative Staph. aureus | | Capsule positive Staph. aureus | |
|---|---|---|---|---|
| | ISB | Milk | ISB | Milk |
| Amp- | 0.198 ± 0.018 | 0.104 ± 0.009 | 0.188 ± 0.019 | 0.12 ± 0.005 |
| icillin | (0.125-0.25) | (0.063-0.125) | (0.125-0.25) | (0.063-0.125) |
| Cep- | 0.188 ± 0.019 | 0.167 ± 0.018 | 0.188 ± 0.019 | 0.25 |
| halo- | (0.125-0.25) | (0.125-0.25) | (0.125-0.25) | |
| thin | | | | |
| Peni- | 0.033 ± 0.004 | 0.031 ± 0.005 | 0.035 ± 0.004 | 0.022 ± 0.002 |
| cil- | (0.025-0.05) | (0.013-0.05) | (0.025-0.05) | (0.013-0.025) |
| lin G | | | | |
| Spi- | 15 ± 1.5 | 25.52 ± 4.82 | 15.83 ± 1.49 | 25 ± 3.77 |
| ramy- | (10-20) | (6.25-50) | (10-20) | (12.5-50) |
| cin | | | | |

4 Effect of tea tree oil (Ttoil) on the effectiveness of ampicillin in nutrient medium (ISB) and milk.

FIC=0.5 additive effect, <0.5 potentiation and >2 antagonism. Staph. aureus A-C are capsule negative and Staph. aureus D-F are capsule positive.

Ampicillin FIC

| | | | | | | | | Fre | |
|---|---|---|---|---|---|---|---|---|---|
| | | | | | | | | quen- | |
| | | | | | | | | cy of | |
| | | | | | | | | po- | |
| | | | | | | | | ten- | Mean |
| | | | | | | | | tion* | val- |
| | | SaA | SaB | SaC | SaD | SaE | SaF | tia- | ue** |
| Ttoil | ISB | 0.063 | 0.125 | 0.125 | 0.125 | 0.25 | 0.25 | 6/6 | 0.14/ |
| | Milk | 0 | 2 | 0 | 2 | 0 | 0 | − | 0.13 |
| | | | | | | | | (2/6) | 2 |

*Ratio of potentiation or ratio of the antagonistic activity with “−” sign. The same applies to Tables 5-7 hereinafter.
**Geometric mean/mean - where appropriate; the same applies to Tables 5-7 hereinafter.

5 Effect of tea tree oil on the potency of activity of cephalothin in broth (ISB) and milk.

FIC=0.5 denotes an additive effect, <0.5 denotes potentiation and >2 denotes antagonism.

Staph. aureus A-C are capsule negative and Staph. aureus D-F are capsule positive.

### Cephalothin FIC

Cephalothin FIC

| | | | | | | | | Fre | |
|---|---|---|---|---|---|---|---|---|---|
| | | | | | | | | quen- | |
| | | | | | | | | cy of | |
| | | | | | | | | po- | |
| | | | | | | | | ten- | Mean |
| | | | | | | | | tion* | val- |
| | | SaA | SaB | SaC | SaD | SaE | SaF | tia- | ue** |
| Ttoil | ISB | 0.063 | 0.125 | 0.125 | 0.063 | 0.5 | 0.25 | 6/6 | 0.44/ |
| | Milk | 0 | 0 | 0 | 0 | 0 | 0.5 | 1/6 | 0.13 |
| | | | | | | | | | 0.5 |

*Ratio of potentiation or ratio of the antagonistic activity with “−” sign. The same applies to Tables 5-7 hereinafter.
**Geometric mean/mean - where appropriate; the same applies to Tables 5-7 hereinafter.

6 Effect of tea tree oil on the activity of penicillin G in nutrient medium (ISB) and milk.

FIC=0.5 denotes additive effect, <0.5 denotes potentiation and >2 denotes antagonism.

Staph. aureus A-C are capsule negative and Staph. aureus D-F are capsule positive.

### Penicillin G FIC

Penicillin G FIC

| | | | | | | | | Fre | |
|---|---|---|---|---|---|---|---|---|---|
| | | | | | | | | quen- | |
| | | | | | | | | cy of | |
| | | | | | | | | po- | |
| | | | | | | | | ten- | Mean |
| | | | | | | | | tion* | val- |
| | | SaA | SaB | SaC | SaD | SaE | SaF | tia- | ue** |
| Ttoil | ISB | 0.063 | 0.125 | 0.125 | 0.25 | 0.125 | 0.25 | 6/6 | 0.14/ |
| | Milk | 0 | 0 | 0 | 2 | 0 | 0 | − | 0.13 |
| | | | | | | | (1/6) | 2 | |

7 Effect of tea tree oil on the activity of spiramycin in nutrient medium (ISB) and milk.

Staph. aureus A-C are capsule negative and Staph. aureus D-F are capsule positive.

### Spiramycin FIC

Spiramycin FIC

| | | | | | | | | Fre | |
|---|---|---|---|---|---|---|---|---|---|
| | | | | | | | | quen- | |
| | | | | | | | | cy of | |
| | | | | | | | | po- | |
| | | | | | | | | ten- | Mean |
| | | | | | | | | tion* | val- |
| | | SaA | SaB | SaC | SaD | SaE | SaF | tia- | ue** |
| Ttoil | ISB | 0.063 | 0.063 | 0.125 | 0.063 | 0.25 | 0.125 | 6/6 | 0.1/ |
| | Milk | 0.5 | 0 | 0.5 | 0 | 0 | 0.5 | 3/6 | 0.09 |
| | | | | | | | | | 0.5 |

II. Typical Recipes or Compositions for Pressurised Gas Foam Preparations

1. Aqueous Suspension of Tea Tree Oil Combined With an Antibiotic

| Component | Proportion [%] | Range [%] |
|---|---|---|
| Tea tree oil | 0.5 | 0.01-20.0 |
| Antibiotic | 5.0 | 0.1-15.0 |
| Foaming agent | 2.0 | 0.1-10.0 |
| Emulsifier | 3.0 | 0.1-20.0 |
| Antioxidant | 0.5 | 0.01-5.0 |
| Preservative | 0.5 | 0.01-2.0 |
| Water | 63.5 | 10.0-90.0 |
| Propellant gas | 25.0 | 1.0-40.0 |
| Total | 100.0 | |

2. Oily Suspension of Tea Tree Oil Combined with an Antibiotic

| Component | Proportion [%] | Range [%] |
|---|---|---|
| Tea tree oil | 0.5 | 0.01-20.0 |
| Antibiotic | 5.0 | 0.1-15.0 |
| Foaming agent | 5.0 | 0.1-10.0 |
| Emulsifier | 5.0 | 0.1-20.0 |
| Antioxidant | 0.5 | 0.01-5.0 |
| Preservative | 0.5 | 0.01-2.0 |
| Oily carrier | 58.5 | 10.0-90.0 |
| Propellant gas | 25.0 | 1.0-40.0 |
| Total | 100.0 | |

3. Booster (Foam Composition), Aqueous, With Tea Tree Oil on its Own

| Component | Proportion [%] | Range [%] |
|---|---|---|
| Tea tree oil | 0.5 | 0.01-20.0 |
| Foaming agent | 2.0 | 0.1-10.0 |
| Emulsifier | 3.0 | 0.1-20.0 |
| Antioxidant | 0.5 | 0.01-5.0 |
| Preservative | 0.5 | 0.01-2.0 |
| Water | 68.5 | 10.0-90.0 |
| Propellant gas | 25.0 | 1.0-40.0 |
| Total | 100.0 | |

4. Booster, Oily, With Tea Tree Oil on its Own

| Component | Proportion [%] | Range [%] |
|---|---|---|
| Tea tree oil | 0.5 | 0.01-20.0 |
| Foaming agent | 5.0 | 0.1-10.0 |
| Emulsifier | 5.0 | 0.1-20.0 |
| Antioxidant | 0.5 | 0.01-5.0 |
| Preservative | 0.5 | 0.01-2.0 |
| Oily carrier | 63.5 | 10.0-90.0 |
| Propellant gas | 25.0 | 1.0-40.0 |
| Total | 100.0 | |

1) Use of a combination of active agents consisting of a bactericidally active substance and a plant extract containing terpene for the production of a veterinary medical preparation for the treatment of microbially caused diseases.

2) Use of a combination of active agents according to claim 1 for the production of a veterinary medical preparation for treating mastitis.

3) Use of a combination of active agents according to claim 1 for the production of a veterinary medical preparation for treating metritis.

4) Use of a combination of active agents according to one of claims 1 to 3, characterised in that the bactericidally or bacteriostatically active agent is an antibiotic.

5) Use of a combination of active agents according to claim 4, characterised in that the antibiotic is an active substance selected from among the aminopenicillins, cephalosporins, benzylpenicillins and/or macrolide antibiotics.

6) Use of a combination of active agents according to one of claims 1 to 5, characterised in that the antimicrobially active agent is an active substance selected from among penicillin G, ampicillin, amoxycillin, bacampicillin, gentamycin, tobramycin, neomycin, kanamycin, framycetin, streptomycin, chloramphenicol, florphenicol, thiamphenicol, oxacillin, cloxacillin, methicillin and percolate from Radix Umckaloabo.

7) Use of a combination of active agents for the preparation of a pharmaceutical composition for topical use according to one of claims 1 to 6, characterised in that the bactericidal agent is an active substance selected from among tetracycline erythromycin, fusidic acid, nebacetin, gentamycin, clindamycin, framycetin, neomycin, chloramphenicol, oxytetracyclin and/or a sulphonamide.

8) Use of a combination of active agents according to claim 6, characterised in that the bactericidal agent is an active substance selected from among ampicillin, cephalothin, penicillin G and/or spiramycin.

9) Use of a combination of active agents according to one of claims 1 to 8, characterised in that the terpene-containing plant extract is an extract from plant parts from one or more plants of the Myrtaceae family.

10) Use of a combination of active agents according to claim 9, characterised in that the plant extract is an extract from parts of plants of the Leptospermum and/or Melaleuca group.

11) Use of a combination of active agents according to claim 9 or 10, characterised in that the plant extract is an extract from parts of plants selected from among Melaleuca alternifolia (tea tree) and/or Melaleuca leucadendra.

12) Use of a combination of active agents according to one of claims 1 to 11, characterised in that the pharmaceutical composition takes the form of a pressurised foam unit (booster preparation).

13) Use of a combination of active agents according to one of claims 1 to 11 in the form of a booster preparation, characterised in that the pharmaceutical composition takes the form of a pressurised foam unit with tea tree oil on its own and is applied immediately after the administration of an antibiotic for enhanced distribution of the active substance and hence for increased activity.

14) Use of a combination of active agents according to one of claims 1 to 13 for preparing a pharmaceutical composition for the treatment of agricultural animals such as sheep, goats, horses, pigs and cattle as well as small animals such as dogs, cats and rabbits.
